(19) United States
(12) Patent Application Publication
(10) Pub. No.: US 2005/0015074 A1
(43) Pub. Date: Jan. 20, 2005

(54) MEDICAL CONNECTOR, FLUID DELIVERY SYSTEM, AND METHOD OF USE THEREFOR

(76) Inventor: Frederick W. Trombley III, Gibsonia, PA (US)

Correspondence Address: GREGORY L BRADLEY;MEDRAD INC, ONE MEDRAD DRIVE, INDIANOLA, PA, 15051, US

(21) Appl. No.: 10/604,402

(22) Filed: Jul. 17, 2003

(51) Int. Cl. A61M 25/16

(52) U.S. Cl. 604/533

(57) ABSTRACT

A medical connector device defines a first passage and a second passage, and includes a first connector and a second connector. The first connector is capable of connecting to a fluid container. The second connector is capable of connecting to a fluid conduit. The first passage extends into the fluid container, and allows air to be vented into the fluid container. The second passage extends between the first connector and the second connector, and allows for the transfer of fluid between the fluid container and the fluid conduit. The connector body may be incorporated into a fluid delivery system including a fluid container, such as a syringe, and a fluid conduit, such as a connector tubing.

### BACKGROUND OF INVENTION

The present invention relates to a medical connector device for connecting a fluid container to a fluid conduit in a fluid delivery system, and to methods of use for the connector device and fluid delivery system.

Millions of infusion procedures are conducted throughout the world on a daily basis. A myriad of different fluids may be administered to a patient to serve a variety of different purposes: parenteral feeding fluids to nourish, saline to hydrate, analgesics to suppress pain, therapeutic drugs to treat disease, contrast medium to image the body, etc. Many of these infusion procedures require the use of an infusion pump similar to that disclosed in US Pat. Application Publication No. 2002/20177821, filed on Jan. 25, 2002, the disclosure of which is incorporated herein by reference. Infusion pumps of this type generally administer fluid from two primary types of containers: IV bags and bottles.

When a fluid is drawn from an air-tight container, such as a bottle, a vacuum is created inside of the respective container. The vacuum, which exists in the region of the container that does not contain fluid, prohibits additional fluid from being drawn from the container. When using IV bags and bottles as a container in infusion procedures, an apparatus known as a vented spike, similar to that disclosed in U.S. Pat. No. 4,128,098, the disclosure of which is incorporated herein by reference, may be used to connect the IV bag or bottle to the IV tubing and alleviate the problem.

Vented spikes generally comprise two passages. The first passage allows for the flow of gas into the fluid container. The second passage allows for the flow of fluid from the fluid container, such as an IV bag or bottle, into a fluid conduit connected, for example, to a patient. The vented spike generally draws fluid from the fluid container, but fills the vacuum that is created in the fluid container by allowing air to be drawn into the fluid container.

Syringes can also operate as effective fluid containers in infusion procedures, but due to many of the difficulties in drawing fluid from the fluid dispensing end of a syringe, traditional vented spikes are incompatible with syringes. Generally, the fluid dispensing end of IV bags and bottles is covered by a membrane that can be penetrated by a vented spike. Once the vented spike penetrates the membrane, the membrane creates a seal around the vented spike, thereby creating an air-tight connection between the vented spike and the fluid container.

U.S. Pat. No. 5,383,858, which is assigned to the same Assignee as the subject application, the disclosure of which is incorporated herein by reference, discloses a typical syringe that can be used in infusion procedures. This syringe, and all substantially similar syringes, comprise a threaded tip at the fluid dispensing end. Typically, medical syringes do not have a membrane similar to those found on IV bags and bottles. Thus, there is no way for a traditional vented spike to suitably connect with the fluid dispensing end of a syringe.

Furthermore, the relatively small diameter of the fluid dispensing end of a syringe, when compared to the diameter of the fluid dispensing end of IV bags and bottles, heightens the threat of venous air embolism, better known simply as embolism, which is most commonly caused by injection of air into the vascular system. Embolism can cause a variety of different symptoms or conditions: dyspnea, chest pain, tachycardia, hypotension, altered sensorium, circulatory shock, acute respiratory distress, tachypnea, tachycardia, agitation, disorientation, cyanosis, and even death. Specifically regarding infusion procedures utilizing a vented spike in conjunction with a syringe, if air bubbles are vented into the tip of the syringe, the air bubbles can easily be drawn into the fluid passage of a traditional vented spike and into the IV tubing, which leads to the patient.

In addition, the relatively small diameter of syringe tips, in general, is not large enough to accommodate spikes of traditional dimensions. However, traditional spikes cannot simply be made smaller in order to fit inside of the syringe tip, for if the fluid passage of the spike is not large enough, than the flow of fluid through the spike may be unduly restricted.

The foregoing differences between infusion procedures that utilize an IV bag or bottle and infusion procedures that utilize a syringe have created a problem in the marketplace. Infusion pumps such as the one disclosed in US Pat. Application Publication No. 2002/2177821, have traditionally only been compatible with IV bags or bottles. To accommodate syringes, infusion pumps such as the Harvard infusion pump, have been developed. These infusion pumps do not draw fluid from the fluid dispensing end of the syringe, but rather interact directly with the syringe plunger, using a drive mechanism to advance the plunger and to expel fluid from the syringe.

As a result, if medical institutions plan on using several different types of fluid containers in infusion procedures, they must purchase two different types of infusion pumps. To escape the extra cost of purchasing an additional infusion pump, many medical institutions simply purchase one type of infusion pump.

Clearly the current technology for connecting a fluid conduit to a fluid container imposes undue costs on medical institutions with regard to infusion procedures.

For the foregoing reasons, there is a need for a new apparatus, system, and method, for delivering fluid to a patient in infusion procedures.

### SUMMARY OF INVENTION

The present invention provides a connector device for use with a fluid container and a fluid conduit. The connector device comprises a connector body, the connector body at least partially defines a first passage operable to allow gas flow into the fluid container, and at least partially defines a second passage operable to allow fluid flow from the fluid container to the fluid conduit.

Furthermore, the present invention provides a connector device comprising a connector body for use with a fluid container having a discharge end and a fluid conduit. The connector body at least partially defines a first passage that is disposed at least partially within, and extends beyond, the connector body. The connector body also at least partially defines a second passage that is disposed at least partially within the connector body, the second passage operable to allow fluid to flow from the fluid container to the fluid conduit. In addition, the connector body comprises a first connector, which is adapted to connect to a discharge end of a fluid container. The connector body also comprises a second connector, which is adapted to connect to the fluid conduit.

In a preferred embodiment, the connector body of the connector device is adapted to connect to a syringe having a discharge injection section, wherein the discharge injection section comprises a tapered conical section, a substantially cylindrical forward connector portion formed with internal screw threads, and an injection nozzle. The first connector of the connector body interacts with the internal screw threads of the syringe to form a luer connection. The first passage, at least partially defined by the connector body, extends within the injection nozzle and into the tapered conical section so that air can be vented into the syringe as fluid is drawn from the syringe. The second connector of the connector body operatively connects to IV tubing so that the second passage, which is at least partially defined by the connector body, allows fluid flow from the syringe to the IV tubing.

By another preferred embodiment of the present invention, the first passage, which is at least partially defined by the connector body, comprises a rigid vent, which also at least partially defines the first passage, wherein the vent extends beyond the first connector of the connector body.

In another preferred embodiment, the first passage is at least partially defined by a bore in the connector body. The second passage is also at least partially defined by a bore in the connector body. In such an embodiment, the connector body can be co-molded.

In yet another preferred embodiment, the connector body is comprised of at least two connected, substantially coaxial bodies. The at least two connected, substantially coaxial bodies can be placed side-by-side so that the first passage is at least partially defined by a different connector body than that which at least partially defines the second passage.

According to another aspect of the present invention, a fluid delivery system is provided for use with a fluid container having a discharge end, and a fluid conduit. The fluid delivery system comprises a medical infusion device, and a fluid conduit capable of connecting to the medical infusion device and a fluid container having a discharge end. The fluid delivery system also comprises a connector device comprising a connector body at least partially defining a first passage disposed at least partially within, and extending beyond, the connector body, and into the discharge end of the fluid container.

In another aspect of the present invention, the first passage is operable to allow gas to flow into the fluid container without drawing fluid from the fluid container into the first passage. The connector body is also designed to allow fluid flow from the fluid container to the fluid conduit.

In a preferred embodiment of the fluid delivery system, the connector body of the connector device is adapted to connect to a syringe having a discharge injection section, wherein the discharge injection section comprises a tapered conical section, a substantially cylindrical forward connector portion formed with internal screw threads, and an injection nozzle. The first connector of the connector body interacts with the internal screw threads of the syringe to form a luer connection. The first passage, which is at least partially defined by the connector body, extends within the injection nozzle and into the tapered conical section so that air can be vented into the syringe as fluid is drawn from the syringe. The second connector of the connector body operatively connects to IV tubing, so that the second passage, which is at least partially defined by the connector body, allows fluid flow from the syringe to the IV tubing. An infusion pump interacts with the IV tubing to draw fluid from the syringe and into the IV tubing. The IV tubing is operatively connected to a patient to allow the infusion of the fluid into the patient.

According to yet another aspect of the present invention, a method of delivering fluid is provided. This method includes the steps of placing a connector, which at least partially defines a first passage, and a second passage, in operative connection with a fluid conduit. Further, the method includes the step of placing the fluid conduit in operative connection with a medical infusion device, and the step of connecting the connector to a fluid container. The connector should be connected so that the fluid conduit is in fluid connection with the fluid container through the second passage, and so that the first passage extends beyond the connector body and within the fluid container to permit the flow of gas into the fluid container. In addition, the method also includes the step of placing the fluid conduit in operative connection with a patient so that fluid may be delivered through the fluid conduit to the patient, and the step of activating the medical infusion device to draw fluid from the fluid container, through the second passage, through the fluid conduit, and to the patient.

The principles, operation, and features of the present invention may be better understood with reference to the drawings and the accompanying description.

### BRIEF DESCRIPTION OF DRAWINGS

FIG. 1 illustrates a cross-sectional view of a currently available vented spike.

FIG. 2 illustrates a cross-sectional view of a connector device of the present invention.

FIG. 3 illustrates the connector device of FIG. 2 connected to a fluid container having a discharge end.

FIG. 4 illustrates a cross-sectional view of another embodiment of the connector device of the present invention.

FIG. 5 illustrates an embodiment of a fluid delivery system of the present invention, including a fluid container having a discharge end, a fluid conduit, a connector device, and an infusion device.

### DETAILED DESCRIPTION

As used herein, the term “fluid container” refers to a receptacle such as a carton, jar, can, syringe, bag, or bottle, in which fluid is held or carried. As used herein, the term “fluid conduit” refers to a pipe, canal, tube, channel, or passage for conveying fluid. As used herein, the term “infusion pump” refers to infusion devices used in administering or infusing fluids to patients.

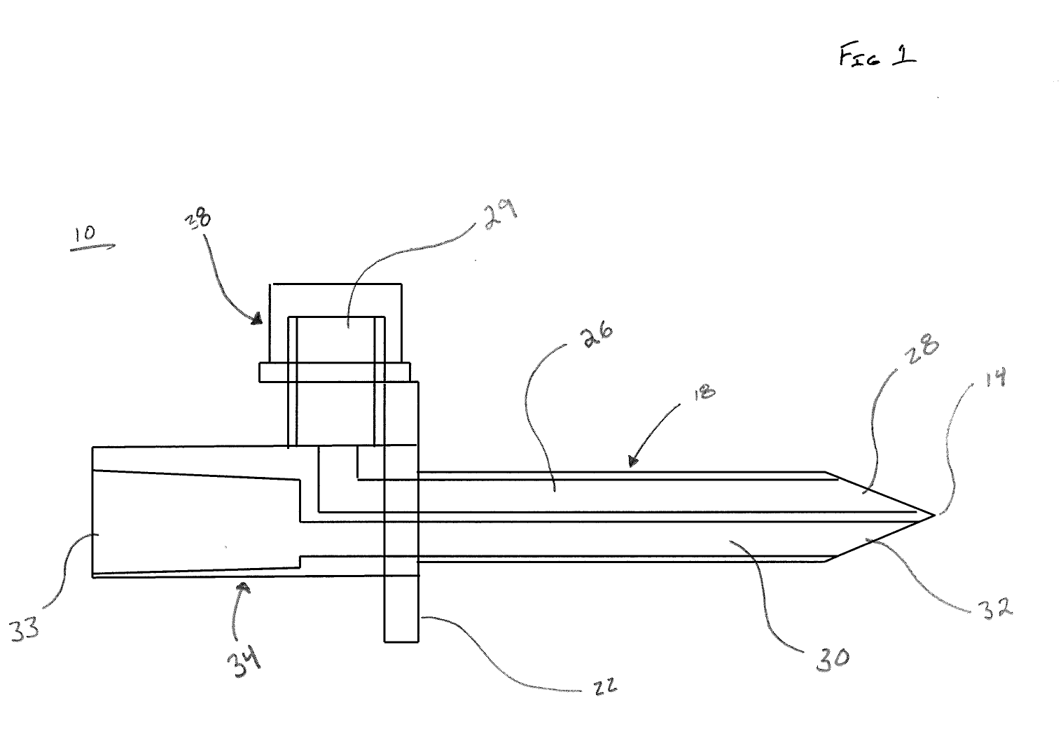

Referring now to the drawings, FIG. 1 illustrates a currently available vented spike. A connector 10 includes a spike 18 that enables penetration of a membrane covering a discharge end of a fluid container. The tip 14 of the spike 18 punctures the membrane, thereby allowing the spike 18 to slide into the fluid container. The crosspiece 22 is designed to allow only the spike 18 to slide into the fluid container. Generally, the spike 18 slides into the fluid container until the membrane covering the discharge end of the fluid container is in contact with the crosspiece 22, which enables an air-tight seal to be formed around the spike 18.

The spike 18 comprises an air passage 26 and a fluid passage 30. The air passage 26 is disposed within the spike 18, and is operable to allow air to flow out of the discharge end 28 of the air passage 26, and into the fluid container. The fluid passage 30 is disposed within the spike 18, and operable to allow fluid to flow into the receiving end 32 of the fluid passage 30 from the fluid container.

Both passages extend into the lower portion 34 of the connector 10. The lower portion 34 of the connector 10 is capable of connecting to IV tubing. Generally, the IV tubing slides onto the lower portion 34 of the connector 10 to create an air-tight seal.

The air inlet 38 is connected to the lower portion 34 of the connector 30 and the crosspiece 22. The air inlet 38 houses part of the air passage 26, and is operable to allow air to flow into the air passage 26 through the receiving end 29 of the air passage 26.

The connector 10 allows fluid to be drawn from a fluid container, such as a bag or bottle (not shown), into the receiving end 32 of the fluid passage 30. Fluid flows through the spike 18 in the fluid passage 30, passing through the lower portion 34 of the connector 10, and out the discharge end 33 of the fluid passage 30 into the IV tubing.

The connector 10 also allows air to pass into the receiving end 29 of the air passage 26 through the air inlet 38. The air passage 26 allows the air to pass through the lower portion 34 of the connector 10 and through the spike 18 to the discharge end 28 of the air passage 26, where the air is discharged into the fluid container.

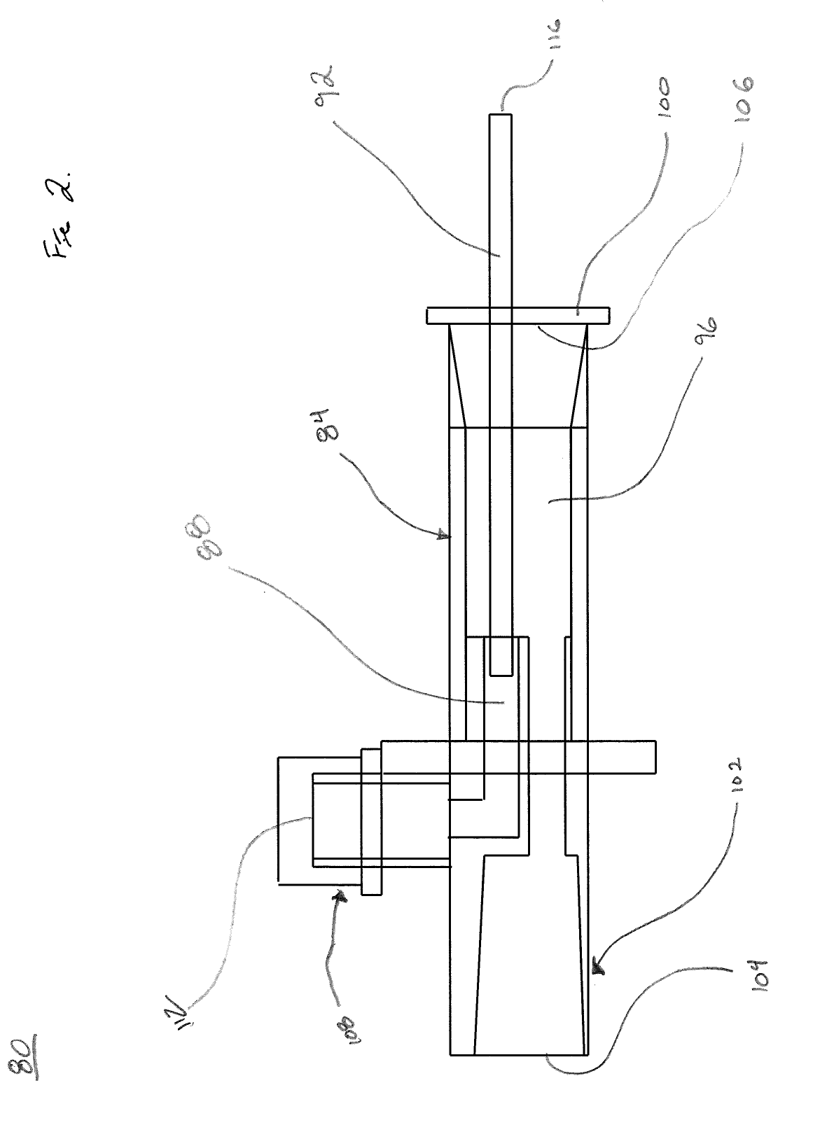

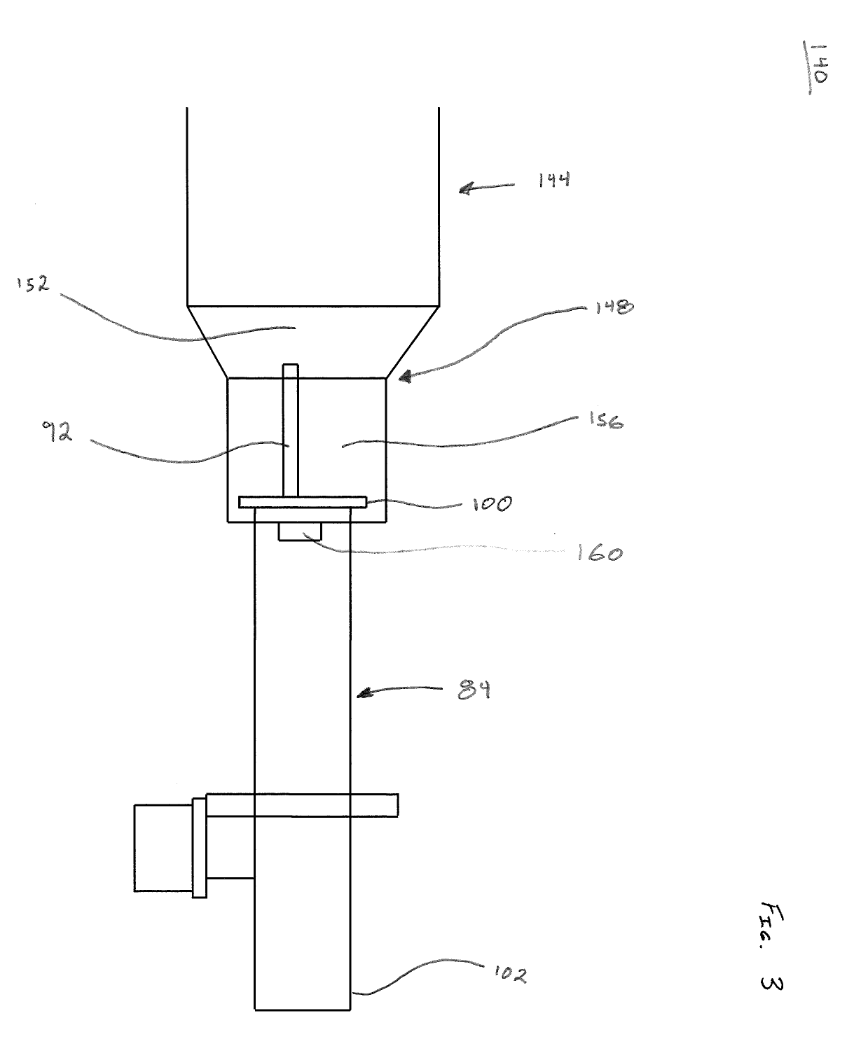

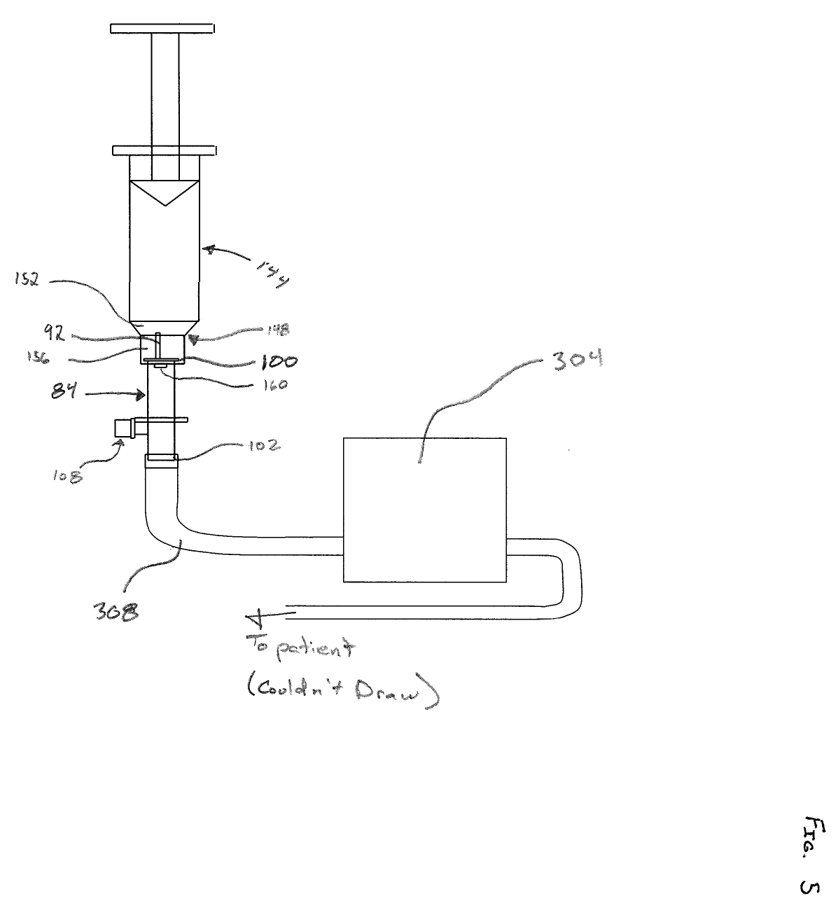

FIG. 2 illustrates a connector device 80 of the present invention. The connector device 80 comprises a connector body 84. The connector body 84 has two connectors, a first connector 100 and a second connector 102. The first connector 100 is capable of connecting to a fluid container (shown in FIG. 3). The second connector 102 is capable of connecting to a fluid conduit (shown in FIG. 5).

The connector body 84 also partially defines a first passage 88 and a second passage 96. The first passage is further partially defined by an gas inlet 108, which is connected to, or integral with, the connector body 84.

Furthermore, the first passage 88 receives gas from the receiving end 112 of the first passage 88 and passes the gas through the first passage 88 to a vent 92. The vent 92 partially defines the first passage 88, and extends the first passage 88 beyond the first connector 100 and into a fluid container. Gas is discharged from the vent 92 at the discharge end 116. One skilled in the art will recognize that the vent 92, while currently an element of this embodiment, is not necessary. As long as gas is appropriately discharged into the fluid container, this embodiment of the present invention will function properly. The connector device 80 could reasonably comprise a co-molded member that defines the first passage 88, and extends beyond the connector body 84.

The second passage 96 receives fluid at the receiving end 106 of the second passage 96, and passes the fluid through the connector body 84 to the discharge end 104 of the second passage 96 to discharge fluid into the fluid conduit.

FIG. 3 illustrates the connector device and the fluid container, such as a syringe 144, in a connected condition. The connector body 84 of FIG. 2 is shown connected to a syringe 144. The connector body 84 comprises the same second connector 102 as shown in FIG. 2.

The syringe 144 is substantially similar to the syringe disclosed in U.S. Pat. No. 5,383,858, which is assigned to the same Assignee as the subject application, the disclosure of which is incorporated herein by reference. One skilled in the art will recognize that this embodiment of the present invention will work with virtually any syringe having a threaded connector portion. One skilled in the art will further recognize that this embodiment will work with virtually any fluid container as long as the first connector 100 is adapted to interact with the discharge end of the fluid container. For example, in another embodiment, the fluid container may be simply connected by relying on a friction fit to maintain the connection between the fluid container and first connector 100, in which case the first connector would be substantially similar to the second connector 102.

The syringe 144 comprises a discharge injection section 148, which has a tapered conical portion 152, a forward connector portion 156, and an injection nozzle 160. The forward connector portion 156 has internal screw threads, and forms the male portion of a luer lock connection. The first connector 100 is the female portion of a luer lock connection, and can be rotated to interact with the forward connector portion 156 of the syringe 144 to form a luer lock connection.

It is notable to mention that the vent 92 extends into the syringe 144, through the injection nozzle 160, past the forward connector portion 156, and into the tapered conical portion 152 of the syringe 144. This is to assure that the gas bubbles emitted by the vent 92 are not drawn back down through the connector body 84, thereby resulting in an injection of gas into the patient, which could cause embolism.

Fluid is drawn from the injection nozzle 160 of the syringe 144 into the second passage 96 of the connector body 84. Gas is discharged into the tapered conical portion 152 of the syringe 144 through the vent 92.

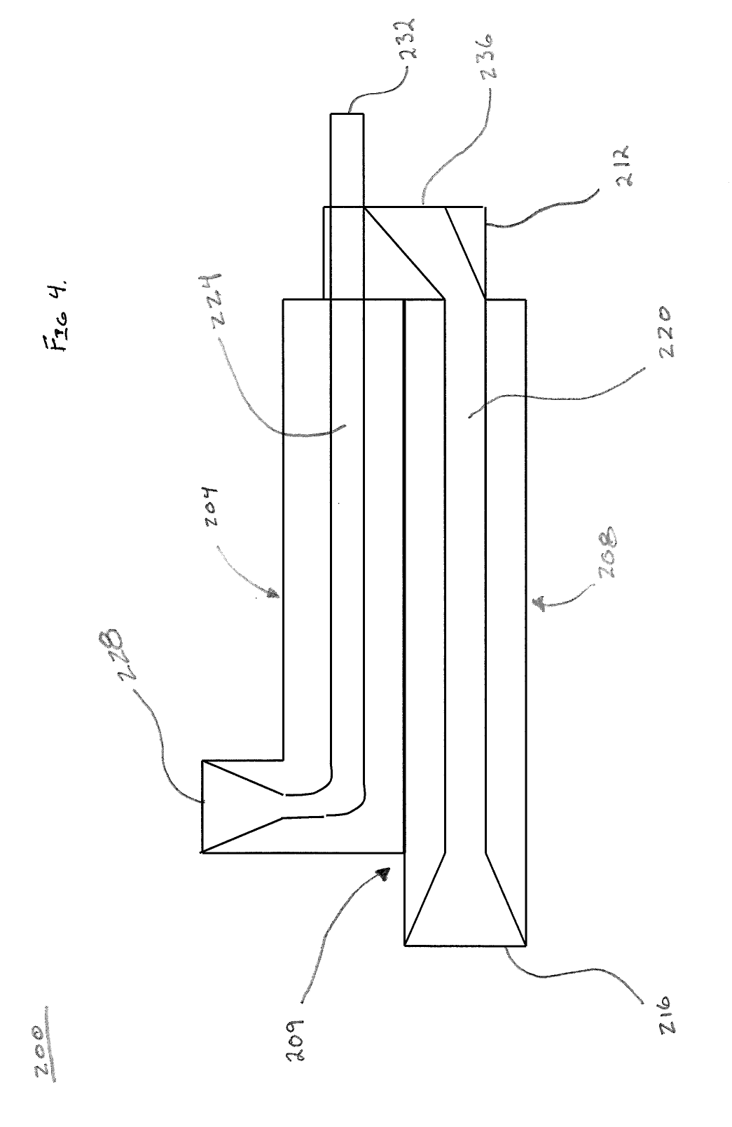

FIG. 4 illustrates a further embodiment of a connector device of the present invention. In this embodiment, the connector device 200 is comprised of two connected coaxial bodies 204 and 208, which together form a single connector body 209. The connector device 200 has a first connector 212 and a second connector 216. The first connector 212 is capable of connecting the connector 200 to a fluid container. The second connector 216 is capable of connecting the connector 200 to a fluid conduit.

The first connector body 204 partially defines a first passage 224 having a receiving end 228 of the first passage 224 and a discharge end 232 of the first passage 224. The first passage 224 is for receiving gas through the receiving end 228 of the first passage 224 and allowing gas to flow into the fluid container through the discharge end 232 of the first passage 224.

The second connector body 208 partially defines a second passage 220 having a receiving end 236 of the second passage 220. The second passage 220 is for receiving fluid from the fluid container through the receiving end 236 of the second passage 220 and allowing the fluid to flow through the second passage 220 into the fluid conduit, which is connected to the second connector 216.

FIG. 5 illustrates an embodiment of a fluid delivery system 300 of the present invention. Herein, the syringe 144 of FIG. 3 is operatively connected to IV tubing 308 by the connector body 84 of FIG. 2. The forward connector portion 156 of the syringe 144 is connected to the first connector 100 of the connector body 84, and the IV tubing 308 is connected to the second connector 102 of the connector body 84.

Once again, it is notable to mention that the vent 92 extends into the injection nozzle 160, past the forward connector portion 156, and into the tapered conical portion 152 of the discharge injection section 148 of the syringe 144. This is to assure that the gas bubbles emitted by the vent 92 are not drawn back down through the connector body 84, and into the IV tubing 308, thereby resulting in an injection of gas into the patient, which could cause embolism.

An infusion pump 304 draws fluid from the syringe 144 by interacting with the IV tubing 308. The infusion pump 304 is substantially similar to the infusion pump disclosed in US Pat. Application Publication No. 2002/20177821, the disclosure of which is incorporated herein by reference. One skilled in the art will recognize that this embodiment of the present invention could easily be adapted to work effectively with virtually any infusion pump.

The infusion pump 304 interacts with the IV tubing 308 to draw fluid from the syringe 144 through the injection nozzle 160, and into the connector body 84. The fluid passes through the connector body 84 from the first connector 100 to the second connector 102, and into the IV tubing 308, which is operatively connected to a patient. This enables the fluid to pass through the IV tubing 308 to the patient.

Since syringes are generally air-tight, if even a nominal amount of fluid is drawn from the syringe 144, a vacuum is created in the syringe 144, thereby making it difficult to draw additional fluid from the syringe 144. This embodiment of the present invention alleviates that problem by accepting air from the ambient in through the gas inlet 108 and passing it through the connector body 84 to be emitted from the vent 92 into the tapered conical portion 152 of the syringe 144.

Although the present invention has been described in detail in connection with the above examples, it is to be understood that such detail is solely for that purpose, and that variations can be made by those skilled in the art without departing from the spirit of the invention. The scope of the invention is indicated by the following claims rather than by the foregoing description. All changes to the present invention that fall within the meaning and range of equivalency of the claims are to be embraced within their scope.

1. An imaging system comprising:
A primary gradient coil assembly; and
A shield coil assembly connected in series to said primary gradient coil assembly, said shield coil assembly comprising:
a first gradient shield coil;
a second gradient shield coil connected in parallel to said first gradient shield coil;
a pair of voltage rails in communication with said first gradient shield coil and said second gradient shield coil;
a first subcircuit in communication with said first gradient shield coil; and
a second subcircuit in communication with said second gradient shield coil said first subcircuit and said second subcircuit independently adjustable such that the currents through said first gradient shield coil and said second gradient shield coil may be independently adjusted.

2. An imaging system as in claim 1 further comprising
at least one additional gradient shield coil connected in parallel to said first gradient shield coil and said second gradient shield coil.

3. An imaging system as in claim 1 wherein said first subcircuit and said second subcircuit are adjusted such that said first gradient shield coil has a resistance equal to said second gradient shield coil.

4. An imaging system as in claim 1 wherein:
said first gradient shield coil comprises a plurality of first shield winding turns; and
said second gradient shield coil comprises a plurality of second shield winding turns, said plurality of second shield winding arms having a non-identical number of turns as said plurality of first shield winding turns.

5. An imaging system as in claim 1 wherein:
said first gradient shield coil comprises a plurality of first shield winding turns and a plurality of winding gaps, each of said plurality of winding gaps formed between consecutive turns of said plurality of first shield winding turs; and
said second gradient shield coil comprises a plurality of second shield winding turns, each of said plurality of second shield winding turns positioned within on of said winding gaps.

6. An imaging system as in claim 5 wherein said plurality of first shield winding turns and said plurality of second shield winding turns are positioned within a single winding plane.

7. An imaging system as in claim 1 wherein said shield coil assembly comprises a plurality of winding turns formed in an asymmetrical pattern.

8. An imaging system as in claim 1 wherein:
said first gradient shield coil comprises a plurality of first shield winding turns forming a first subsoil;
said second gradient shield coil comprises a plurality of second shield winding turns forming a second sub-coil, said second sub-coil positioned linearly adjacent to said first sub-coil and position within a single winding plane.

9. An imaging system comprising:
A primary gradient coil assembly; and
A shield coil assembly surrounding said primary gradient coil assembly, said shield coil assembly comprising;
a first gradient shield coil; and
a second gradient shield coil connected in parallel to said first gradient shield coil;
a pair of voltage rails in communication with said first gradient shield coil and said second gradient shield coil;
a first subcircuit communication with said first gradient shield coil; and
a second subcircuit in communication with said second gradient shield coil, said first subcircuit and said second subcircuit independently adjustable such that the currents through said first gradient shield coil and said second gradient shield coil may be independently adjusted;
wherein said shield coil assembly comprises a plurality of winding turns formed in an asymmetrical pattern.

10. An imaging system as in claim 9 further comprising
at least one additional gradient shield coil connected in parallel to said first gradient shield coil and said second gradient shield coil.

11. An imaging system as in claim 9 wherein said first subcircuit and said second subcircuit are adjusted such that said first gradient shield coil has a resistance equal to said second gradient shield coil.

12. An imaging system as in claim 9 wherein:
said first gradient shield coil comprises a plurality of first shield winding turns; and
said second gradient shield coil comprises a plurality of second shield winding turns, said plurality of second shield winding turns having a non-identical number of turns as said plurality of first shield winding turns.

13. An imaging system as in claim 9 wherein:
said first gradient shield coil comprises a plurality of first shield winding turns and a plurality of winding gaps, each of said plurality of winding gaps formed between consecutive turns of said plurality of first shield winding turns, and
said second gradient shield coil comprises a plurality of second shield winding turns, each of said plurality of second shield winding turns positioned within on of said winding gaps.

14. An imaging system as in claim 9 wherein:
said first gradient shield coil comprises a plurality of first shield winding turns forming a first sub-coil;
said second gradient shield coil comprises a plurality of second shield winding turns forming a second sub-coil, said second sub-coil positioned linearly adjacent to said first sub-coil and position within a single winding plane.

15. An imaging system as in claim 13 wherein said plurality of first shield winding turns and said plurality of second shield winding turns are positioned within a single winding plane.

16. (Cancelled)

17. A method of reducing the fringe field generated by a primary gradient coil assembly comprising:
running a first current through a first gradient shield coil connected in parallel to the primary gradient coil assembly;
running a second current through a second gradient shield coil connected in series to the primary gradient coil assembly, said second gradient shield coil connected in parallel to said first gradient shield coil,
adjusting said first current and said second current independently to minimize the fringe field.

18. (Cancelled)

19. (Cancelled)

20. A method as described in claim 17, wherein said first current and said second current are passed through an equal number of winding turns.

21. A method as described in claim 17, wherein said first gradient shield coil and said second gradient shield coil share a single winding plane.
